(19) United States
(12) Patent Application Publication
(10) Pub. No.: US 2005/0043531 A1
(43) Pub. Date: Feb. 24, 2005

(54) Process for preparing cefepime

(76) Inventors: Vijay Kumar Handa, Hyderabad (IN); Anand G. Kamat, Hyderabad (IN); Meenakshisunderam Sivakumaran, Hyderabad (IN)

Correspondence Address: Jay R Akhave, 845 Pomello Dr, Claremont, CA, 91711, US

(21) Appl. No.: 10/688,606

(22) Filed: Oct. 17, 2003

(30) Foreign Application Priority Data
Aug. 21, 2003 (IN) 669/CHE/2003

(51) Int. Cl. C07D 501/14

(52) U.S. Cl. 540/224

(57) ABSTRACT

A novel process is disclosed for the preparation of Cefepime, a cephalosporin antibiotic, using novel new intermediates of the general Formula, 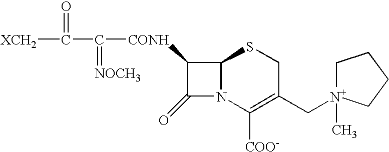 where X represents Bromine or Chlorine atom This process comprises the step of cyclizing the bromo or chloro intermediate with thiourea to produce Cefepime of high purity. A process to prepare bromo or chloro intermediate comprising the acylation of 7-Amino-3-[(1-methyl-1-pyrrolidinium) methyl]-3-cephem-4-carboxylate with 4-halo-2-methoxyimino-3-oxobutyric acid halide is also described.

### CROSS REFERENCE TO RELATED APPLICATIONS

Indian Patent Application Filing Date Aug. 21, 2003
Application No. 669/CHE/2003
Status Not issued

### BACKGROUND OF THE INVENTION

Cefepime, also known as 7-[(Z)-2-(2-Amino-4-thiazolyl)-2-(methoxyimino)acetamido]-3-[(1-methyl-1-pyrrolidinium)methyl]-3-cephem-4-carboxylate is a useful broad spectrum antibiotic cephalosporin and has the chemical structure of Formula I

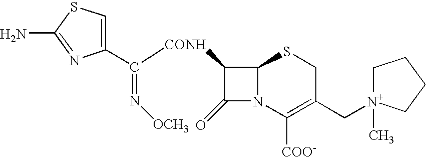

Cefepime and its preparation has been first disclosed in U.S. Pat. 4,406,899. Two reaction schemes have been discussed in this patent to prepare Cefepime. Both of these schemes make use of the protecting groups that require additional blocking and deblocking steps. Furthermore, the exemplified process makes use of a chromatographic purification step to obtain Cefepime zwitterion.

U.S. Pat. No. 4,754,031 describes a process where 2-(2-amino-4-thiazolyl)-2-methoxyiminoacetic acid is activated by reacting with methanesulfonyl chloride to form an anhydride for acylation of 7-Amino-3-[(1-methyl-1-pyrrolidinium)methyl]-3-cephem-4-carboxylate to obtain Cefepime. Although this process does not use protecting groups but it requires column chromatography as a purification method which is not practical in manufacturing.

U.S. Pat. 5,594,129 describes preparation of Cefepime wherein acid chloride hydrochloride of the Formula, 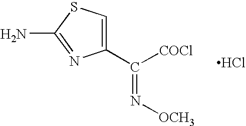
has been used for the N-acylation of 7-Amino-3-[(1-methyl-1-pyrrolidinium)methyl]-3-cephem-4-carboxylate under anhydrous conditions. The use of the same acid chloride hydrochloride in aqueous conditions for N-acylation to prepare Cefepime has been demonstrated in the U.S. Pat. 5,594,130. In both of these U.S. patents, the preparation of the desired acid chloride hydrochloride involves first the conversion of syn-2-(2-amino-4-thiazolyl)-2-methoxyiminoacetic acid to the corresponding hydrochloride salt which is then treated with chlorinating agent under specifically defined reaction conditions to obtain the syn-isomer of 2-(2-amino-4-thiazolyl)-2-methoxyiminoacetyl chloride hydrochloride that contains less than about 5% of the undesirable anti-isomer which may affect the subsequent acylation reaction to obtain the antibiotic.

Cephalosporins have been prepared in literature through an alternate method in which the amino group in the cephem nucleus is first acylated with 4-halo-2-methoxyimino-3-oxobutyric acid and the thiazolyl ring formation is subsequently effected with thiourea. However, there is no such report yet to date for preparing Cefepime through this route. This procedure of preparing a cephalosporin compound is described in the present invention to obtain highly pure Cefepime.

### DETAILED DESCRIPTION OF THE INVENTION

The present invention relates to 7-(4-halo-3-oxo-2-methoxyiminobutyrylamino)cephalosporin compounds of the general Formula II 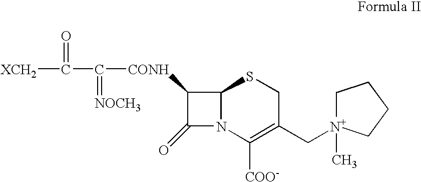
where X represents Bromine or Chlorine atom
and also to a process for preparing Cefepime of Formula I 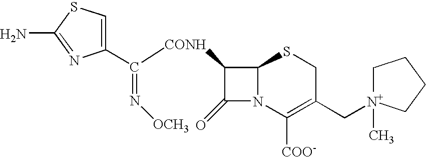
as well as its salts and hydrates, which comprises reacting the above compounds of Formula II with thiourea and converting Cefepime of Formula I into a hydrate of the said salt.

According to the present invention, the intermediate compounds of Formula II are prepared by N-acylation of 7-amino-3-[(1-methyl-1-pyrrolidinium)methyl]-3-cephem-4-carboxylate of Formula III 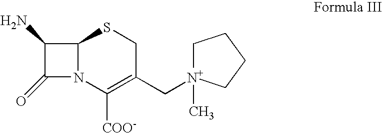
or its HX salt wherein HX is HCI, Hi or H₂SO₄, with halogenated carboxylic acid of the general Formula IV 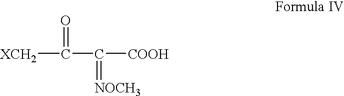
where X represents Bromine or Chlorine atom

4-(Bromo or Chloro)-2-methoxyimino-3-oxobutyric acid of Formula IV is prepared in high purity and good yield starting from tert-butylacetoacetate as per the procedure described in the U.S. Pat. 5,095,149. tert-Butylacetoacetate has been prepared from tert-butylacetate as given in Organic Synthesis Coll. Vol. V, p-156. This is converted into the corresponding acid chloride of Formula V 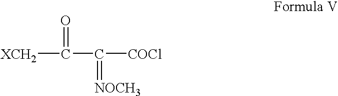
where X represents Bromine or Chlorine atom
by reacting with halogenating agents such as phosphorous oxychloride, phosphorous pentachloride, oxalyl chloride etc and the acid chloride thereby produced may be isolated prior to acylation with cephalosporin compound or may be generated in situ and used as such. The acid chloride formation is conducted in an inert organic solvent such as chloroform, methylene chloride, acetonitrile or the like and most preferably the reaction is carried out in methylene chloride at a temperature of −25° C. to −15° C.

The cephalosporin compound of Formula IlI and its HX salt, which is substantially free from Δ²-isomer, may be prepared by the general procedure described in the U.S. Pat. 5,594,131.

The cephalosporin compound, 7-amino-3-[(1-methyl-1-pyrrolidinium)methyl]-3-cephem-4-carboxylate, which is preferably available as its hydrochloride salt, may advantageously be silylated in an inert organic solvent to form an in situ solution of the soluble silylated derivative. It is important to add sufficient silylating agent to solubilize the cephalosporin compound of Formula IlI before treating it with acid chloride of Formula V. Silylating agents which may be used are, for example, hexamethyldisilazane, trimethylchlorosilane, N,N′-bis(trimethylsilyl)urea, N-(trimethylsilyl)acetamide, N,O-bis(trimethylsilyl)acetamide, tert-butyldimethylchlorosilane or the like and most preferably N-(trimethylsilyl)acetamide may be used.

Suitable solvents which may be used in the acylation process are all inert organic solvents in which the silylated derivative of cephalosporin compound of Formula IlI is soluble, for example, toluene, tetrahydrofuran, acetone, acetonitrile, methylene chloride, chloroform or the like and most preferably methylene chloride may be used. Soluble silylated derivative is then treated with the acid chloride of Formula V, preferably with one molar equivalent, and most preferably with a slight excess of the acid chloride. The silylation of cephalosporin compound of Formula III is completed at about 15° C. to 30° C. while the N-acylation is advantageously carried out at −30° C. to −10° C.

After N-acylation is complete, as ascertained by the known detection methods reported in the art, water is added to the reaction mixture to precipitate 7-(4-halo-3-oxo-2-methoxyiminobutyrylamino)cephalosporin compound of the general Formula II, which is isolated by filtration. The halo intermediates of Formula II and their preparation from Cephalosporin compound of Formula III constitutes the inventive part of the present invention to prepare Cefepime.

The reaction of halo intermediates of Formula II with thiourea to prepare Cefepime, in accordance with the present invention is preferably carried out in a solvent such as ethanol, acetone, tetrahydrofuran, N,N-dimethylformamide, water and mixture thereof and preferably aqueous acetone is used. The reaction is generally carried out at a temperature range of 20° C. to 40° C. and preferably at room temperature. Thereafter, when it is desired to prepare Cefepime dihydrochloride monohydrate, the reaction mass after cyclization with thiourea is treated with sufficient amount of hydrochloric acid. The resulting reaction mixture is then diluted with water miscible appropriate solvent such as acetone to ensure the crystallization of the desired Cefepime dihydrochloride monohydrate form.

The Cefepime dihydrochloride monohydrate thus obtained is substantially free from anti-isomer and Δ²-isomer. The present process provides control of the stereochemical configuration of methoxyimino isomer and the Δ³-double bond of cephalosporin nucleus without the need to separate undesirable cephalosporin by-product by chromatography. Another advantage of present invention is the use of acid chloride of Formula V wherein the simple chloride ion is the leaving group and thus avoids unusual and sometimes complex leaving groups described in the art.

The examples below illustrate our invention without limiting the scope of the invention. The examples are described as two stage processes where the first stage forms the preparation of the inventive intermediates, and the second stage is their conversion to Cefepime dihydrochloride monohydrate.

### Example-1

Stage-I:

Step-A: Silylation of 7-AMINO-3-[(1-METHYL-1PYRROLIDINIUM)METHYL]-3-CEPHEM-4-CARBOXYLATE HYDROCHLORIDE (Solution A)

To a suspension of 7-amino-3-[(1-methyl-1-pyrrolidinium)methyl]-3-cephem-4-carboxylate hydrochloride (10 g, 0.03 mol) in methylene chloride (100 ml) at 20-25° C., N-trimethylsilylacetamide solution (containing 26.72 g N-trimethylsilylacetamide, 0.20 mol) was added and stirred for 1 hour to obtain a clear solution. This solution was cooled to −25° C. to −20° C. until use.

Step-B: Preparation of 4-BROMO-2-METHOXYIMINO-3 OXOBUTYRYLCHLORIDE (Solution B)

To a suspension of phosphorous pentachloride (7.5 g; 0.036 mol) in methylene chloride (62 ml), 4-bromo-2-methoxyimino-3-oxobutyric acid (7.73 g, 0.035 mol) was added in small lots over a period of 10 minutes, while maintaining the temperature between −25° C. and −20° C. The reaction mass was stirred at −25° C. to −20° C. until the starting material's absence was noted with TLC (30 minutes). The reaction mass was then washed with water (23 ml) to remove inorganic impurities and by-products. This solution was used as such in the next step.

Step-C: Preparation of 7-(4BROMO-2-METHOXYIMINO-3-OXOBUTYRAMIDO)-3-[(1- METHYL-1-PYRROLIDINIUM)METHYL]-3-CEPHEM-4-CARBOXYLATE (Bromo Intermediate)

Solution B was added to solution A, while maintaining the temperature between −25° C. and −20° C. over a period of about 10 minutes and the reaction mass was stirred for 1 hour at this temperature. Thereafter cold water (50 ml, 5° C.) was added and the reaction mass was stirred at 2-5° C. for 1 hour. The product thus obtained was filtered, washed with methylene chloride (20 ml) and dried to obtain the bromo intermediate as its hydrochloride salt (13.2 g). The structure of this compound was confirmed by spectroscopic data.

¹H NMR (300 MHz) (DMSO-d₆) δ: 2.11 (m, 4H), 2.94 (s, 3H), 3.45 (m, 1H), 3.59 (m, 3H), 3.66 & 4.05 (2d, each 1H), 4.05 (s, 3H), 4.30 & 4.61 (2d, each 1H), 4.86 (s, 2H), 5.33 (s, 1H), 5.91 (dd, 1H),9.55 (d, 1H).

IR (KBr) cm⁻¹: 1785, 1714, 1678, 1610

MASS (Positive ion Mode) : 503, 505 [M+1]; 525, 527 [M+Na] corresponding to ⁷⁹Br and ⁸¹Br isotopes.

Stage-II:

Preparation of Cefepime Dihydrochloride Monohydrate

Thiourea (0.31 g, 0.0040 mol) was added to a suspension of bromo intermediate (2.0 g, 0.0037 mol, as obtained above) in a mixture of acetone (20 ml) and water (10 ml) at 20-25° C. The reaction mass was stirred at 20-25° C. for 2 hours. The pH was adjusted to 6.7 using triethylamine (1 ml) and the reaction mass was stirred for 10 minutes. Thereafter, reaction mass was cooled and concentrated hydrochloric acid (2.8 ml) was added at 5-8° C. followed by acetone (60 ml) The resulting slurry was cooled and stirred at 0-5° C. for 1 hour. The product thus obtained was filtered, washed with acetone (2×5 ml) and dried to obtain 1.47 g of Cefepime dihydrochloride monohydrate having HPLC purity 99.42%.

¹H NMR (300 MHz) (DMSO-d₆) δ: 2.10 (m, 4H), 2.94 (s, 3H), 3.45 (m, 1H), 3.59 (m, 3H), 3.66 & 4.04 (2d, each 1H), 3.93 (s, 3H), 4.31 & 4.61 (2d, each 1H), 5.33 (d, 1H), 5.89 (dd, 1H), 6.88 (s, 1H), 8.51 (b, 2H), 9.83 (d, 1H).

IR (KBr) cm⁻¹: 1773, 1730, 1658, 1634

MASS (Positive ion Mode) : 481 [M+1]⁺; 503 [M+Na]⁺

### Example-2

Stage-I:

Preparation of 7-(4-CHLORO-2-METHOXYIMINO-3-OXOBUTYRAMIDO)-3-[(1-METHYL-1-PYRROLIDINIUM)METHYL]-3-CEPHEM-4-CARBOXYLATE (Chloro Intermediate)

4-Chloro-2-methoxyimino-3-oxobutyric acid (6.2 g, 0.0345 mol) was added to a suspension of phosphorous pentachloride (7.5 g, 0.0360 mol) in methylene chloride (62 ml) in small lots over a period of 10 minutes while maintaining temperature between −25° C. and −20° C. The reaction mass was stirred at −25° C. to −20° C. until completion of the reaction (˜1 hour) and then washed with cold water (23 ml, 5° C.). The resulting acid chloride is reacted with silyalted 7-amino-3-[(1-methyl-1-pyrrolidinium)methyl]-3-cephem-4-carboxylate hydrochloride as per the procedure given in Example-1 to obtain the chloro intermediate as its hydrochloride salt. The structure of this compound was confirmed by spectroscopic data.

¹H NMR (300 MHz) (DMSO-d₆) δ: 2.11 (m, 4H), 2.94 (s, 3H), 3.45 (m, 1H), 3.60 (m, 3H), 3.68 & 4.06 (2d, each 1H), 4.05 (s, 3H), 4.34 & 4.60 (2d, each 1H), 4.86 (s, 2H), 5.33 (s, 1H), 5.90 (dd, 1H), 9.58 (d, 1H)

IR(KBr) cm⁻¹: 1785, 1717, 1682, 1609

MASS (Positive ion Mode): 459, 461 [M+1] corresponding to ³⁵CI and ³⁷CI isotopes.

Stage-II:

Preparation of Cefepime Dihydrochloride Monohydrate

Thiourea (0.92 g, 0.012 mol) was added to a suspension of chloro intermediate (4.0 g, 0.008 mol, as obtained above) in a mixture of acetone (40 ml) and water (20 ml). The reaction mass was stirred at 23-30° C. till completion of reaction (˜6 hours).

Thereafter, reaction mass was cooled and concentrated hydrochloric acid (1.2 ml) was added at 5-8° C. followed by addition of acetone (92 ml). The product thus obtained was filtered, washed with acetone (2×10 ml) and dried to obtain 3.4 g of Cefepime dihydrochloride monohydrate having HPLC purity 99.19%.

1. A process for the manufacture of Cefepime of the Formula I 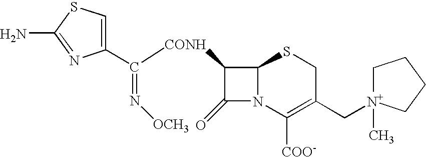
as well as its salts and hydrates, comprising the steps of reacting a compound of general Formula II with thiourea to obtain Cefepime of Formula I or its salts or its hydrates 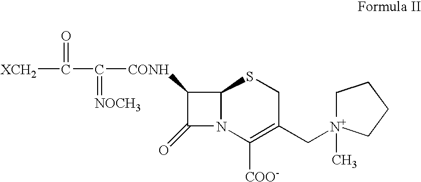
where X represents Bromine or Chlorine atom

2. A process according to claim 1 wherein the X in said compound of Formula II represents a bromine atom.

3. A process according to claim 1 wherein the X in said compound of Formula II represents a chlorine atom.

4. A process according to claim 1 wherein a compound of Formula II is prepared by acylating an amine compound of Formula III or its salt 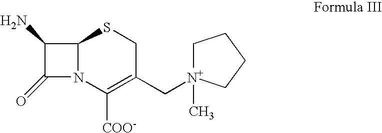
with a halogenated carboxylic acid of the general Formula IV or a reactive derivative thereof 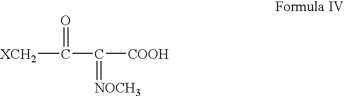
where X represents Bromine or Chlorine atom

5. A process according to claim 1 wherein a compound of Formula II is prepared by acylating an amine compound of Formula III or its salt 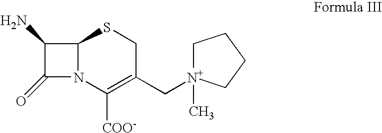
with an acid chloride of the halogenated carboxylic acid of the general Formula V. 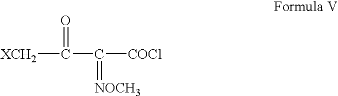
where X represents Bromine or Chlorine atom

6. A process according to claim 1 wherein Cefepime of Formula I is a dihydrochloride monohydrate.

7. Novel intermediate compounds of the general Formula II, useful in the preparation of Cefepime. 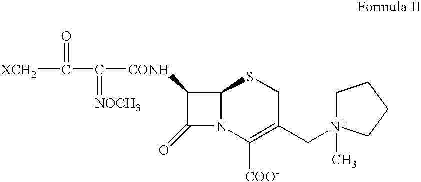
where X represents Bromine or Chlorine atom
